(19) United States
(12) Patent Application Publication
(10) Pub. No.: US 2011/0046450 A1
(43) Pub. Date: Feb. 24, 2011

(54) Visiblade - a transparent disposable surgical retractor based on fis ("form in situ") technology

(76) Inventor: Theodore A. Blaine, Barrington, RI (US)

Correspondence Address: Theodore A Blaine, M.D, 5 Payne Road, Barrington, RI, 02806, US

(21) Appl. No.: 12/583,574

(22) Filed: Aug. 24, 2009

(51) Int. Cl. A61B 1/32

(52) U.S. Cl. 600/206

(57) ABSTRACT

The subject instruments are surgical retractors known as the Visiblade™ that can be anatomically “customized” for each surgical procedure. The retractors are single blade instruments which are comprised of plastic composite and semi-liquid (pliable) acrylic resin materials that can be formed in-situ to a desired configuration. Once configured, the retractor blade is exposed to a light source where the acrylic resin attains a rigid state. The retractors are single use (sterile), translucent, radiolucent and non-abrasive. They improve visualization, reduce risk of infection and minimize the need for multiple pre-formed retractor instruments during surgical procedures. The retractors are intended to be disposed after use and are made of materials that can be recycled.

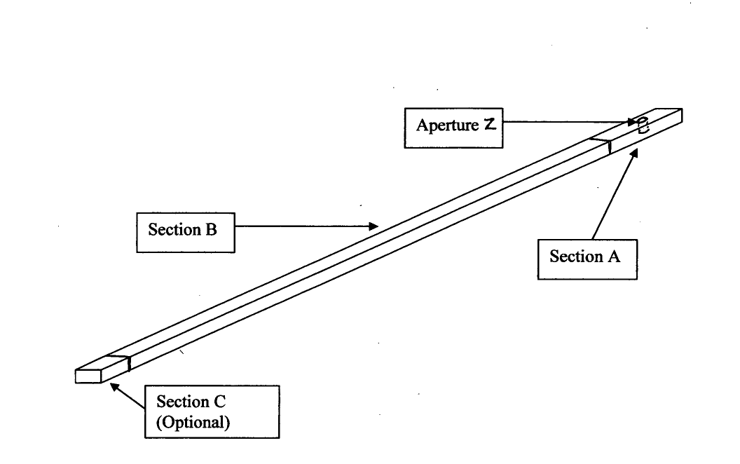

### CROSS-REFERENCE TO RELATED APPLICATIONS

Provisional Patent Application No. 61/135,631 by Theodore A. Blaine M. D dated Jul. 23, 2008

### BACKGROUND

In the performance of a surgical procedure, a surgeon makes one or more surgical incisions in a patient. To obtain greater visibility and unhindered access to underlying anatomical structures during surgery, the surgeon uses retractor(s) to dilate or reflect tissues including skin, muscle, fat, bone and any or all other tissues. (See US Patent Classification, Class 600—Surgery, Subclass 201—Retractor Subject matter having one or more surfaces for holding tissues or organs out of the field of view of the observer during the treatment of interior bodily orifices that are either surgically formed in, or occur naturally within, the body). In general, the greater the visibility and access the surgeon has to the surgical site, the greater the probability that a successful outcome will result.

Visibility in the surgical field. The purpose of retractors is to allow visualization within a surgical wound by moving aside (retracting) adjacent tissues. Even with the advent of “minimally invasive surgery” (often referred to as “MIS”), most surgical procedures require an exposed surgical field for the surgeon to successfully perform the procedure. While they maintain an exposed surgical field, most retractors are opaque and non-radiolucent, meaning that when used as intended, they can and do reduce visibility. This can be especially problematic in MIS procedures, where the exposed field is smaller.

Variability in currently available surgical retractors. Retractors and other surgical instruments should not obstruct either visual or physical access to the site. Different surgical protocols typically require different styles of retractors. In order to accomplish this, there are a variety of retractors currently available. Each surgical facility maintains a large stock of these various instruments and retractors which require inventory control, cleaning, storage and sterilization processes.

Avoiding injury and infection. The exposed surgical fields must be maintained while also protecting and avoiding injury to these tissues. Injury may include but is not limited to direct traumatic injury, thermal or abrasive injury, and the introduction of bacteria and organisms which may cause infection. Because of their rigidity and material properties (metal), retractors can be abrasive to bones, tissue, implantable devices and prostheses.

As with all surgical procedures, it is critical to take all precautions to ensure the sterility of the operating field. In most cases, retractors are made of surgical steel and are intended to be used repeatedly. This means that on-site sterilization equipment (e.g., autoclave) and procedures are required. However, on-site sterilization and reuse of retractors may not be effective to ensure sterility, as sterilization equipment may not function properly, or sterilization procedures may not be followed.

Form-in-situ and “blade” technology. Typical retractors are made from pre-formed surgical steel and include one or more smooth projections, generally termed “blades” which retain an area of tissue adjacent to an incision. Blades may be straight or curved, open or dosed end, and vary in length and width. The retracting blades can be provided in a wide array of configurations. Retractors are available from various producers who carry an extensive offering of variable configurations. The surgeons selects from this wide array of devices based on their need in any particular operation.

Due to the fact that retractors are pre-formed, surgeons must have several shapes and sizes on hand because the structure, physique, and location of the wound or operable condition varies with each patient. While there are some malleable or moldable retractors available in the market that can be adjusted to “fit” the patient, such retractors generally do not hold their shape or form under the forces experienced in orthopedic and other surgical procedures.

Disposal and Re-use. Like many surgical instruments, most surgical retractors are not intended to be disposable. As they are available in a wide variety of shapes, sizes and configurations, medical facilities typically maintain many retractors for use during surgery. Retractors are intended to be (and are) used again and again in different patients, thus requiring sterilization. Because retractors come into contact with bodily fluids and tissues, reuse carries a higher risk to infection even when industry standard sterilization practices are followed.

### BRIEF SUMMARY OF INVENTION

The Visiblade™ is a surgical instrument designed to manipulate a patients tissues in the same manner as currently available surgical retractors. The Visiblade™ is a single blade instrument constructed of clear plastic composite and acrylic materials. The significant portion of the Visiblade™ is comprised of a pliable, light cured acrylic resin can be molded (i.e., Formed In-Situ) by a surgeon according to his needs based on the individual anatomy/physiology of the patient. When the surgeon is satisfied with the form of the blade, it is then exposed to a light source where the blade is cured by reaction to light in the appropriate spectrum. Once cured, the blade attains sufficient hardness to hold its shape during surgery. The retractor is intended to be disposed after use, minimizing the risk of infection to each patient. The Visiblade™ is designed to solve numerous problems associated with industry standard retractors currently available in the market, including: partial surgical field and radiographic view obstruction due to opaque steel, metal or composite composition; abrasiveness on implants or natural anatomical structures; non-conformity with patient anatomy/physiology; and sterility due to multiple use.

### BRIEF DESCRIPTION OF THE SEVERAL VIEWS OF THE DRAWING

The drawing depicts a general perspective view of the Visiblade™ surgical retractor system in rectangular configuration. Section A is the plastic composite base composed of Lexan™ (or an alternative polycarbonate plastic). Within Section A is an aperture labeled as “z” that is used to connect the retractor to a retaining device. Section B is the main section of the Visiblade™ composed of a clear dual cure acrylic resin material that is pliable in uncured state and rigid in the cured state. Section C is an optional part of the instrument that would serve as a liquid dam on the operative end should the composition of the acrylic resin in Section B requires such feature.

### DETAILED DESCRIPTION OF INVENTION

The Visiblade™ surgical retractor system is comprised of a dual cure chemical mechanism embodied in a “plastic” composite carrier body.

As depicted in Drawing 1, the Visiblade™ is a single blade instrument. It may be manufactured in a variety of dimensions and configurations based on customer preferences and demand. However, the base form is intended to be rectangular.

The primary feature which distinguishes the Visiblade™ from other surgical retractors is its composition. Specifically, the Visiblade™, as its name suggests, is composed of clear plastic composite and acrylic resin materials that are transparent so that the retractor does not obstruct the surgeon's view of the surgical field.

From a structural standpoint, the Visiblade™ is comprised of two or more sections. The first section is designed as a liquid dam at the retractor end and is made of solid Lexan™ (or an alternative polycarbonate plastic). This section provides a base that serves as a handle or gripping surface for the surgeon to hold. The Lexan™ surface contains an aperture by which the Visiblade™ may be connected to a retaining device that will hold it in place during the surgical procedure.

The primary section of the Visiblade™ is longer in length than the first section. This section of the blade comprised of a light cured acrylic resin in semi-liquid form can be manipulated by the surgeon to achieve the desired shape. The chemical composition of the resin may be similar to currently available acrylic resins used in other forms of medical device, non-retractor applications, but it will be specially formulated to optimize blade cure and performance for surgical conditions. The chemistry of the resin is not the subject of this application. However, for the purpose of Visiblade™ use, the resin must be translucent, radiolucent, and pliable prior to curing. Curing time for the resin once it is exposed to the light source must be very short. Once cured, the resin must be high in modulus structure and retain its translucence and radiolucence. The resin will be developed to meet all of the above requirements, as well as applicable FDA standards for medical uses.

Before curing, the resin is in a semi-liquid state and may be contained in a thin plastic sheath, depending on the pre-cured rigidity of the resin (i.e., whether it maintains its shape under STP conditions). To the extent that a sheath is utilized, it may be retained or removed before or after curing, depending on the final formulation of the resin. This section of the Visiblade™ may be adjusted during manufacture to meet the specific needs of the end user, and therefore may be flat, convex or cylindrical. In general, however, the base product design is a flat rectangular section with dimensions (length, width, height) of approximately 11 inches×½ to 1 inches×¼ inches.

Finally, the Visiblade™ may include a third section. This section is a short 1 inch or less solid Lexan™ (or alternative polycarbonate plastic) that is rectangular or curved in shape that is attached to the operative (surgical) end of the primary Visiblade™ section. This section of the instrument is optional and if employed, will serve to provide the surgeon with an additional solid surface to manipulate the retractor into the desired configuration.

As described above, the design of the Visiblade™ surgical retractor is intended to have all the basic characteristics of retractors necessary for major open surgery applications, as well as MIS procedures. However, the design features of the Visiblade™ address many deficiencies and concerns related to currently available surgical retractor instruments and systems. Specifically, the Visiblade™ features, including transparency/translucence, radiolucence, pliability for Form In-Situ use, light cured, post-cure rigidity, sterility and disposability solve problems related to surgical and radiographic field view obstruction, potential damage to implants or other natural anatomic structures, incompatibility with patient physiology/anatomy and risk of infection from multiple use.

The inventor claims as follows:

1. A hand-held surgical retractor instrument referred to as the Visiblade™ that is used to manipulate patient tissues and which is: (a) comprised of a dual cure chemical resin contained in a plastic composite carrier body; (b) translucent; (c) radiolucent; (d) moldable for Form in-Situ configuration appropriate to patient anatomy; (e) light activated to cure the molded retractor blade into a rigid form that retains its shape during surgery; (f) non-abrasive; (g) sterile at point of use; (h) disposable and recyclable; and (i) suitable for manufacture in varying lengths and widths.
The retractor in claim 1(a) is a single blade instrument constructed of clear plastic composite and acrylic material, the substantial portion of which contains a chemical component that is flexible in its uncured state, as opposed to being a single or multiple blade instrument made of pre-formed surgical steel.
The retractor in claim 1(b) is comprised of clear plastic composite and acrylic material that is translucent and will not obscure the surgeon's direct view of important structures in surgery, especially with minimally invasive surgical procedures.
The retractor in claim 1(c) is comprised of clear plastic composite and acrylic material that is radiolucent so that it will not obscure views of important structures under x-ray imaging.
The retractor in claim 1(d) is comprised in significant part of a flexible, pliable acrylic material that allows the surgeon to reshape the blade according to the specific anatomy/physiology of the patient and the surgical procedure.
The retractor in claim 1(e) is comprised in significant part of a flexible, pliable acrylic material that is light activated, converting the flexible molded blade into a rigid instrument that maintains the shape during surgery.
The retractor in claim 1(f) is constructed of plastic composite and acrylic materials that are non-abrasive or minimally abrasive so that it will not damage implants (e.g., orthopedic joint prosthesis), avoiding the potential for wear and failure which may be introduced by scratched bearing surfaces.
The retractor in claim 1(g) is a single use device that is intended to remain in sterile packaging until it is ready to be used in accordance with FDA standards; this reduces the risk of infection to patients arising from reusable steel retractors that may not be property disinfected even when following the current industry standard sterilization processes.
The retractor in claim 1(h) is disposable and is comprised of plastic composite and acrylic material that is recyclable; single use promotes sterility, recyclable materials reduce waste.
The retractor in claim 1(i) is can be manufactured in multiple widths or lengths, as well as base shape (flat, curved or cylindrical) which further enhances the surgeon's ability to select and form the device “in situ” to a desired configuration that best suits the surgeon's needs.
